(19) United States
(12) Patent Application Publication
(10) Pub. No.: US 2016/0338821 A1
(43) Pub. Date: Nov. 24, 2016

(54) APPARATUS AND METHOD FOR PROTECTING A PATIENT'S VASCULAR SYSTEM FROM DEBRIS

(71) Applicant: T. Anthony DON MICHAEL, Bakersfield, CA (US)

(72) Inventor: T. Anthony DON MICHAEL, Bakersfield, CA (US)

(21) Appl. No.: 15/162,203

(22) Filed: May 23, 2016

Related U.S. Application Data
(60) Provisional application No. 62/165,288, filed on May 22, 2015.

(51) Int. Cl. A61F 2/01

(52) U.S. Cl. CPC A61F 2/013; A61F 2002/018

(57) ABSTRACT

A kit and a system for providing long term protection for a patient's vascular system against damage from debris produced in the patient's ascending aorta as a result of a medical procedure involving the patient's heart. The kit and the system include an aortic filter assembly constructed to block debris in the patient's aorta while allowing blood flow through the aorta. The kit includes a shunt adapted to be implanted to form a flow path between an artery downstream of the debris removal tube and a vein and a venous filter adapted to be placed in the vein downstream of the shunt to collect debris flowing from said shunt. The system includes a heart-lung machine.

### BACKGROUND OF THE INVENTION

The present invention relates to an apparatus and procedure for aiding medical treatments in the blood circulation system of a patient, and in particular for preventing the circulation of embolic debris, or blood clots, resulting from such treatments. The invention is primarily, but not exclusively, concerned with providing protection during a period following a surgical procedure performed on the heart, including those for implanting a prosthetic heart valve and for open heart surgery.

### BRIEF SUMMARY OF THE INVENTION

The present invention provides novel appliances that can be safely used on a relatively long term basis, e.g., for several weeks, to prevent debris in the circulatory arising from a surgical procedure on or in the vicinity of the heart from circulating downstream of the surgical site.

### BRIEF DESCRIPTION OF THE DRAWINGS

FIG. 1 is a pictorial view showing a first embodiment of the invention.

FIG. 2 is a view similar to that of FIG. 1 showing a second embodiment of the invention.

### DETAILED DESCRIPTION OF THE INVENTION

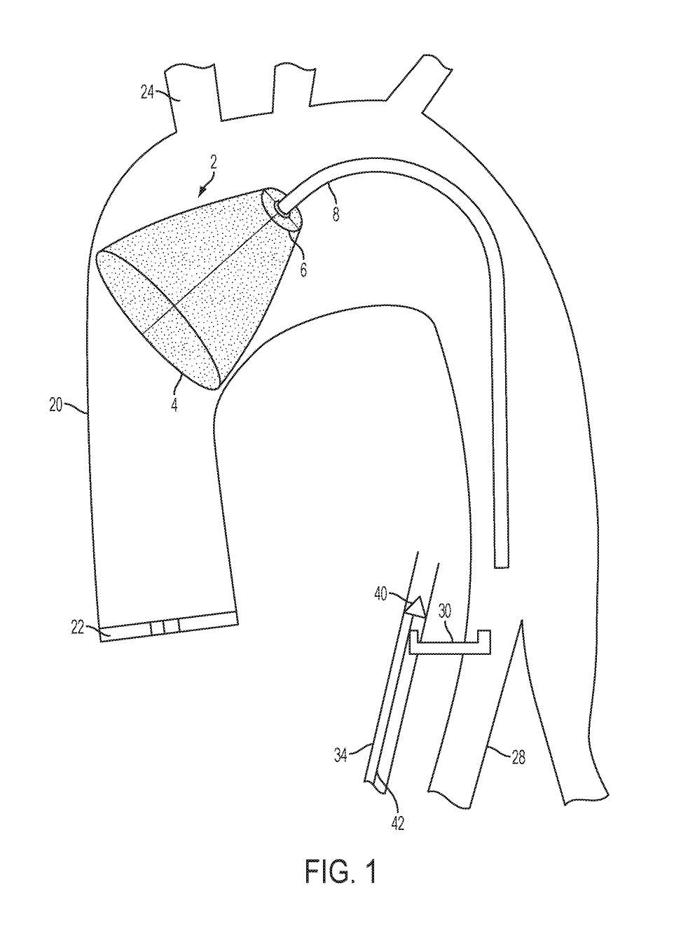

FIG. 1 illustrates one embodiment of a filter 2 constituting one component of a kit according to the invention. Filter 2 may have the form of a filter disclosed in copending U.S. application Ser. No. 13/835,816, particularly in FIGS. 10-12 thereof, the disclosure of which is incorporated herein by reference. Filter 2 is composed of a wire framework, preferably made of a memory metal such as nitinol, and a filter fabric of appropriate pore size to permit blood flow while blocking debris, the filter fabric being bonded to, and supported by, the framework. The framework includes a ring 4 at the open, large diameter end, a ring 6 at the small diameter end, and several longitudinal struts, or ribs, joining rings 4 and 6 together.

Filter 2 has a generally frustoconical structure. In the expanded state of filter 2, the diameter of the small diameter end can be in the range of 18-26 mm and the maximum diameter of the large diameter end can be of the order of 35-40 mm, although the diameter can be larger or smaller to match the diameter of the blood vessel, such as the aorta, in which the filter is to be used.

The large diameter end of filter 2 is formed to have a generally circular shape, or an oval shape with a major diameter of about 40 mm and a minor diameter of the order of 30 mm. This allows the lower end of the filter to better conform to the somewhat oval shape of a normal aorta.

Of course, the dimensions of filter 2 can be varied to conform to aortas having different sizes, for example in children.

Filter 2 has a form defined by an outwardly bowed arcuate generatrix of rotation about the longitudinal axis of filter 2, such that the wall of the filter bows outwardly, as shown in FIG. 1.

Filters composed of a framework of memory metal, e.g. nitinol, wires can be constructed to present a radial expansion/compression ratio of 8:1, or more. They may be held, in a compressed state, in a sheath or tube (not shown) having an inner diameter preferably equal to or greater than ⅛ the desired expanded diameter of the large diameter end of the filter.

The framework may be provided with four struts, or ribs, or may be provided with a different number of struts. The filter framework can have many other configurations depending on the configuration and size of the blood vessel in which the filter is to be employed. The framework can be made of individual wires that are soldered or otherwise secured together.

At the small diameter end, ring 6 encloses filter fabric, but is provided with a central opening that is free of filter fabric for receiving a debris removal tube 8, which tube may have a diameter of at least 5 Fr (3 Fr=1 mm), and preferably 5-6 Fr. Tube 8 is dimensioned to form a sealed connection with the central opening.

Filter 2 and tube 8 may be introduced into an aorta 20 through an appropriate sheath (not shown), as also disclosed in the above-cited U.S. application. The sheath is preliminarily passed through, for example, a femoral artery, a subclavian artery, or a radial artery with the aid of a guidewire (not shown), and then passed into the aorta. Preferably, filter 2 is positioned so that the large diameter end is between the aortic valve 22 and the innominate artery 24, and ring 6 is downstream of ring 4, as shown in FIG. 1.

Catheter 8 can, for example, be a pigtail catheter and is used to drain debris from filter 2 via the central opening in upper end 6. Then, the above-mentioned sheath may be withdrawn. Tube 8 is given a length sufficient to extend through the ascending and descending aortas to a location possibly in or proximal to the femoral artery 28.

The apparatus according to the invention is further provided with a shunt 30, which is a hollow tube that is open at both ends and that may have a U-shaped form. Shunt 30 would be implanted so that its inlet end faces the outlet end of tube 8, while the outlet end of shunt 30 is disposed in a vein 34 that is preferably proximal to femoral artery 28.

The apparatus further includes a second filter 40 that is composed of a framework and a filter fabric similar to, but smaller than, filter 2. Filter 40 may have a form similar to that of one of the filters described and illustrated in U.S. Pat. No. 7,806,906, the disclosure of which is incorporated herein by reference. Filter 40 may have a conical form and the side of filter 40 will be covered with a suitable fabric similar to the fabric of filter 2. Filter 40 is connected to a guide wire 42 which is used to preliminarily position filter 40 in vein 34. Filter 40 may be introduced into vein 34 through a suitable sheath (not shown) in a conventional manner, which sheath is then withdrawn.

Due to the inherently lower pressure in vein 34, debris exiting tube 8 will be drawn into and through shunt 30, where it will be collected by filter 40.

One effect of shunt 30 is to reduce the pressure in the aorta downstream of filter 2. As a result, the pressure in the aorta upstream of filter 2 will be reduced, thereby reducing stress on the patient's heart.

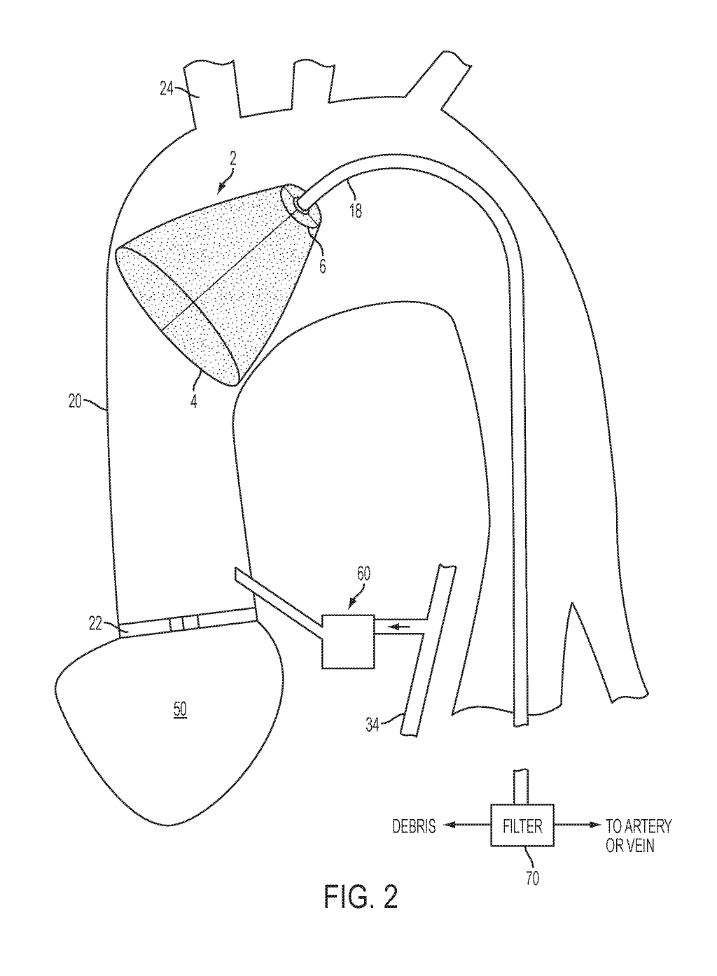

FIG. 2 shows a second embodiment of the invention that may be used during open heart surgery, during which the patient's heart 50 is generally stopped and the ascending aorta may or may not be clamped, and can advantageously remain in place after surgery. Elements identical to those shown in FIG. 1 are given the same reference numerals and will not again be described in detail.

Filter 2 is introduced into the ascending aorta 20 in the manner described above with reference to FIG. 1, generally prior to beginning a surgical procedure. If a clamp is used on the ascending aorta, this clamp will be positioned between aortic valve 22 and filter 2.

A conventional heart-lung machine 60 is used to maintain perfusion to other body organs and tissues while the surgeon works in a bloodless surgical field. The surgeon places a cannula in, for example, the right atrium, vena cava, or femoral vein, 34 to withdraw blood from the body. The cannula is connected to tubing filled with isotonic crystalloid solution. Venous blood that is removed from the body by the cannula is filtered, cooled or warmed, and oxygenated in machine 60, and then returned to the body. The cannula used to return oxygenated blood is usually inserted in the ascending aorta 20, as shown, but it may also be inserted in the femoral artery.

Filter 2 is connected to a debris removal tube 18 that is coupled to filter 2 in the same manner that tube 8 of FIG. 1 is coupled to filter 2, and extends through the ascending and descending aortas and possibly the femoral artery, to a location outside the patient's body. Tube 18 may be introduced along other blood vessel paths, such as a path composed of a subclavian artery and a communicating radial artery.

The proximal end of tube 18, i.e. the end that will be outside of the patient's body, is secured to a filter 70. Filter 70 may be constructed according to principles already well known in the art.

When filter 2 has been introduced to the desired location and deployed, i.e., radially expanded, and the surgical procedure is being performed, debris produced by the procedure will be conveyed, along with blood, into filter 2. A portion of the blood will then pass through the filter mesh, or fabric, that covers the circumference of filter 2 between ends 4 and 6 while the debris, along with some blood, will flow through small diameter end 6 and tube 18 to filter 70. In filter 70, debris will be separated from blood and the filtered blood may then be conducted into an artery, such as the aorta, as shown, or a vein to be returned to the circulatory system.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A kit for providing long term protection for a patient's vascular system against damage from debris produced in the patient's ascending aorta as a result of a medical procedure involving the patient's heart, said kit comprising:
an aortic filter assembly composed of a filter constructed to block debris in the patient's aorta while allowing blood flow through the aorta and past said aortic filter, and a debris removal tube connected to said filter in a manner to receive debris blocked by said filter and to conduct the debris away from the aorta;
a shunt adapted to be implanted to form a flow path between an artery downstream of the aorta and downstream of said debris removal tube and a vein; and
a venous filter adapted to be placed in the vein downstream of said shunt to collect debris flowing from said shunt.

2. A system for providing protection for a patient's vascular system against damage from debris produced in the patient's ascending aorta as a result of a medical procedure involving the patient's heart, said system comprising:
an aortic filter assembly composed of a filter constructed to block debris in the patient's aorta while allowing blood flow through the aorta and past said aortic filter, and a debris removal tube having a distal end connected to said filter in a manner to receive debris blocked by said filter and to conduct the debris away from the aorta; and
a heart lung machine connectable between a vein and an artery of the patient during the medical procedure.

3. The system of claim 2, wherein said tube has a proximal end remote from said distal end and said tube has a length selected to cause said proximal end of said tube to extend out of the patient's body when said filter is positioned in the patient's ascending aorta.

4. The system of claim 3, further comprising an external filter connectable to said proximal end of said tube and constructed to separate debris from blood.
